(19) United States
(12) Patent Application Publication
(10) Pub. No.: US 2014/0272799 A1
(43) Pub. Date: Sep. 18, 2014

(54) YTTRIA-TREATED PORCELAIN VENEER

(71) Applicant: Research Triangle Institute, Research Triangle Park, NC (US)

(72) Inventors: Jeffrey Robert Piascik, Raleigh, NC (US); Brian R. Stoner, Chapel Hill, NC (US)

(73) Assignee: Research Triangle Institute, Research Triangle Park, NC (US)

(21) Appl. No.: 14/212,592

(22) Filed: Mar. 14, 2014

Related U.S. Application Data
(60) Provisional application No. 61/787,571, filed on Mar. 15, 2013.

(51) Int. Cl. A61K 6/02; A61C 13/00; A61C 8/00; A61C 5/10

(52) U.S. Cl. CPC A61K 6/0255; A61C 5/10; A61C 13/0006; A61C 8/0013; A61C 8/005
USPC 433/199.1; 433/167; 433/222.1; 433/201.1; 427/2.29

(57) ABSTRACT

A dental component comprising a modified porcelain veneering coating thereon is provided, wherein the porcelain veneering coating can comprise a plurality of crystalline inclusions. The crystalline inclusions can serve to strengthen the porcelain veneering coating and the dental component as a whole. A method for the preparation of such treated implants is also provided, the method involving providing a dental component; applying a porcelain slurry comprising about 5% or more by weight of an additive capable of forming a crystalline silicate phase to at least a portion of the surface of the dental component to give a coated dental component; and firing the porcelain-coated dental component.

### CROSS-REFERENCE TO RELATED APPLICATION

This application claims priority to U.S. Provisional Patent Application Ser. No. 61/787,571, filed Mar. 15, 2013, the entire contents of which are hereby incorporated by reference.

### FIELD OF THE INVENTION

The invention is related to methods for enhancing the strength and for reducing failure of dental restorations. It is also related to dental restorations wherein one or more materials are functionalized to endow the restorations with enhanced strength.

### BACKGROUND OF THE INVENTION

Prosthetic dental restorations can be direct restorations or indirect restorations. Direct restorations are often known as “fillings,” and require a soft material to be applied to a cavity located in a tooth. The soft material is subsequently cured to give a restored tooth structure. Indirect restorations are generally fabricated before being used within the mouth, and then the finished restoration is bonded to an appropriate structure within the mouth (e.g., existing tooth structure, bone, synthetic implant abutment, etc.). Exemplary indirect restorations include, but are not limited to, bridges, crowns, inlays, onlays, and veneers, etc. The chemical makeup of such indirect restorations can vary.

Restorations comprising a porcelain veneer layered on a core material are often used, particularly where aesthetics are a concern (e.g., to address concerns with the front teeth). Porcelain is a white, translucent ceramic that is applied and fired to a glazed state. Generally, dental labs first construct a core. Subsequently, layers of porcelain are applied to an outer surface of the core, which can then be heated to sinter/solidify the porcelain and create a physical “fit” on the core. Porcelain veneers are advantageous in their ability to mimic the look of natural tooth by the application of multiple layers of varying translucency. Various materials can serve as the core material for such a veneer, including, but not limited to, natural tooth, metal, and/or ceramics. Good adhesion is important in such applications for high retention, prevention of microleakage, and fracture and fatigue resistance. In order to ensure good adhesion between a porcelain veneer and a core material, various methods have been utilized, including, but not limited to, particle abrasion, acid etching, application of bonding agents, and silanation of the core surface.

High strength ceramics such as alumina and zirconia-based ceramics can be particularly advantageous as core materials, as they may provide better fracture resistance and long-term durability than traditional dental materials. While there has been much research directed to enhancing the bonding between the core and the underlying structure, the majority of clinical failures are attributed to chipping or failures associated with the core/porcelain veneer interface. Several such failure modes have been shown for restorations comprising high strength ceramic cores and porcelain coating veneers. Chipping has been observed, which results from a loss of adhesion at the core/veneer interface, created from a mismatch between the coefficients of thermal expansion of the two materials and indicating no chemical bonding between the high strength ceramic core and the porcelain veneer. Veneer failure has also been shown, where cracking is initiated within the porcelain, created from firing and from a mismatch between the coefficients of thermal expansion of the two materials. These failures can range from small chips within the porcelain layer to defects that extend to the core/porcelain veneer interface.

It would be advantageous to better understand the basis for such failures and to provide all-ceramic dental restorations wherein such failures are minimized (i.e., all-ceramic restorations with increased reliability and longevity).

### BRIEF SUMMARY OF THE INVENTION

In one aspect of the present invention is provided a modified dental porcelain material. In certain embodiments, preparation and use of such modified dental porcelain materials are readily compatible with existing ceramic lab and dental processes and protocols. For example, certain modified dental porcelain materials provided herein can be readily prepared by mixing and applied to other dental components using traditional techniques.

Specifically, the modified dental porcelain material, following application and firing, can comprise a high density of crystalline phases throughout. Such crystalline phases, typically present as crystalline inclusions in an otherwise amorphous porcelain material can, in some embodiments, serve to strengthen the material and may advantageously minimize failure modes associated with fractures in the porcelain veneer.

Certain aspects of the invention provide a method of increasing the integrity of a dental restorative, comprising: providing a dental component; applying a porcelain slurry comprising about 5% or more by weight of an additive capable of forming a crystalline silicate phase to at least a portion of the surface of the dental component to give a coated dental component; and firing the coated dental component. The type of dental restorative can vary and may, in some embodiments, comprise a crown, bridge, veneer, inlay, or onlay. The dental component can comprise, for example, an abutment.

According to the methods described herein, the makeup of the porcelain slurry can vary. For example, the porcelain slurry may comprise about 10% or more by weight of the additive capable of forming a crystalline silicate phase. The additive can be, for example, a metal, a metal compound, or a metal salt. One exemplary additive comprises YF₃.

Similarly, the makeup of the dental component can vary. In certain embodiments, the dental component comprises a tetragonally stabilized ceramic. For example, the dental component may comprise a yttria-stabilized ceramic (e.g., YSZ). In some embodiments, the dental component comprises an optionally stabilized zirconia, alumina, titania, or chromium-oxide.

In some embodiments of the methods described herein, each of the applying and firing steps can be performed at least two times. In certain embodiments, the methods can further comprise treating the dental component with a fluorine-containing reagent prior to the applying step.

In another aspect of the present disclosure is provided a dental restorative comprising a dental component and a modified porcelain coating overlying at least a portion of the dental component, wherein the modified porcelain coating comprises a plurality of crystalline inclusions present in an amount of at least about 5% by volume of the porcelain coating. For example, the crystalline inclusions may be present in an amount of between about 5% and about 40% crystalline inclusions by volume. The crystalline inclusions can, in certain embodiments, comprise metal silicate inclusions, including but not limited to, yttrium silicate inclusions.

In yet another aspect of the disclosure is provided a dental restorative comprising a yttria-stabilized zirconia dental component and a modified porcelain coating overlying at least a portion of the dental component, wherein the modified porcelain coating comprises a plurality of yttrium silicate inclusions present in an amount of at least about 2% by volume of the porcelain coating.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIGS. 1A and 1B are depictions of an exemplary dental structure of the present invention, wherein the “X” notations indicate the ceramic (e.g., YSZ) interfaces;

FIG. 2 is a SEM image of a highly polished YSZ material showing areas of yttrium-silicate inclusions;

FIG. 3 shows: (A) a fracture surface of a ZirPress/ZirCAD crown after the cusp chipped off, showing the fracture origin and the fracture path through the veneer; and (B) a micro-CT scan of the mirror image of the same fracture surface, where radio-opaque inclusions (white specs) can be seen on the fracture surface;

FIG. 4 shows: (A) a micro-CT scan of a cross-section of a veneered e-max Ceram ZirCAD plate, where white spots indicate crystalline yttrium silicate inclusions that have migrated through the thickness of the porcelain veneer; and (B) an SEM scan of a cross-section of the same material, showing areas of migrated Y that have formed crystalline defects; and

FIG. 5 shows a schematic illustration of (A) an inclusion present in a veneering ceramic that is a specific defect for early failure (i.e., serving as a point for crack initiation) and (B) a structure with a greater number of inclusions, which can, in some embodiments, increase the overall toughness of the veneering porcelain.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

According to the present disclosure, means for increasing the robustness (e.g., reliability and longevity) of a medical implant are provided. Specifically, with respect to dental restoratives, the present disclosure relates to modifying the composition of a porcelain veneer component by incorporating certain additives therein and to medical implants comprising a porcelain veneer coating having such modifications. Methods and materials to minimize failure modes typically associated with porcelain veneer components of dental implants are described in further detail herein.

The inventors have found that the treatment of certain ceramic core components can result in migration of certain ions. For example, it has been shown that a YSZ material subjected to F plasma treatment undergoes Y ion migration toward the surface of the material. See Piascik et al., Dental Mat. (2011) 27(5): e99-e105; Piascik et al., J. Biomed. Mater. Res. B: Applied Biomat. (2011) 98B(1) 114-119; and PCT Int. App. Pub. No. WO/2013/158836 to Piascik et al,, which are incorporated herein by reference. The inventors have further found that, in untreated YSZ materials, high temperature processing of porcelain veneer layers on the YSZ materials can lead to migration of Y ions through the YSZ and across the YSZ/porcelain interface. Defects/inclusions comprising the Y ions within the porcelain can subsequently form near the ceramic/porcelain interface.

Such inclusions are considered to be flaws, representing potential sources of crack initiation within the porcelain. Although in these relatively small concentrations, these inclusions would be expected to negatively impact failure modes, the inventors have found that inclusions in the porcelain in significantly larger concentrations can surprisingly inhibit fracture propagation within the porcelain and thus may strengthen the porcelain. The disclosure thus additionally relates to methods for specifically forming and enhancing inclusions such that they are present in concentrations sufficient to strengthen and inhibit fracture propagation within the porcelain veneer. Accordingly, in some embodiments, ceramic-based restorations are provided, comprising a high strength ceramic and a modified porcelain material, wherein the porcelain material comprises a plurality of microcrystalline and/or nanocrystalline defects/inclusions,

### I. Definitions

“Dental implant” as used herein means a post (i.e., a dental abutment) anchored to the jawbone and topped with individual replacement teeth or a bridge that is attached to the post or posts. The term is meant to encompass traditional dental implants as well as mini-dental implants. In some cases where the dental abutment is in the form of natural tooth, the dental implant only comprises the implanted replacement tooth or bridge.

“Restorative” or “restoration” as used herein means any dental component used to restore the function, integrity and/or morphology of any missing tooth structure. Examples of restoratives that may be provided according to the methods described herein include, but are not limited to, crowns, bridges, fillings, veneers, inlays and onlays, as well as endodontic devices including endodontic cones and devices for endodontic root perforation repair.

“Orthodontic device” as used herein means any device intended to prevent and/or correct irregularities of the teeth, particularly spacing of the teeth. Orthodontic devices particularly relevant to the present invention include but are not limited to orthodontic brackets.

“Dental component” as used herein encompasses any component of a dental implant or a restorative or an orthodontic device and can even include, in certain embodiments, natural tooth.

“Porcelain” as used herein generally refers to dental porcelain, also known as dental ceramic. The chemical composition of such porcelains is widely variable and they may comprise such components as clay (in the form of kaolin/kaolinate), glass, quartz, feldspar, bone ash, steatite, prtuntse, and alabaster. Dental porcelains can, in some embodiments, contain single metal oxides or various mixtures of metal oxides (e.g., silica, aluminum oxide, calcium oxide, potassium oxide, titanium dioxide, zirconium oxide, tin dioxide, rubidium dioxide, barium oxide, boric oxide, and/or other oxides). Another exemplary component of a dental porcelain in some embodiments is leucite (crystals of a potash-alumina-silica complex). Various materials can be included within a porcelain, for such purposes as enhanced strength. Other exemplary porcelain materials are described, for example, in EP Patent Publication EP 0272745, which is incorporated herein by reference in its entirety.

### II. Modified Porcelains

According to certain aspects of the invention, a method to modify the interaction between two dental components (e.g., a porcelain-based dental component (e.g., a restoration) and a second dental component) is provided. For example, in certain specific embodiments, the porcelain-based dental component comprises a veneer and the second dental component is a ceramic core (e.g., an abutment). This type of dental structure is illustrated in FIG. 1. FIG. 1A is an illustration of a tooth structure, wherein the tooth structure is modified with a ceramic abutment (depicted in white) and the ceramic abutment is coated with a porcelain layer (depicted in dark grey, on the surface of the tooth structure). A cross-section of these layers and the interfaces therebetween is illustrated in FIG. 1B. However, the components can comprise any types of dental components, restoratives, or orthodontic devices as described above. Various means are known for preparing and shaping second dental components. For example, in certain embodiments, computer-aided design and computer-aided manufacturing (CAD/CAM) techniques are used as understood and commonly used in the dental field.

The second dental component advantageously comprises a high-strength ceramic (e.g., a zirconia, alumina, titania, or chromium-oxide-based material which may be unstabilized (i.e., pure) or may comprise a stabilized material, e.g., a fully or partially stabilized ceramic material). Particularly preferred according to the present disclosure are tetragonally-stabilized systems. A stabilized material generally is stabilized by doping with one or more ions that can replace some of the ions in the metal oxide lattice of the ceramic. For example, in specific embodiments, the ceramic may be stabilized with an oxide (e.g., yttrium oxide, magnesium oxide, calcium oxide, aluminum oxide, and/or cerium(III) oxide). In certain specific embodiments, the second dental component comprises yttria-stabilized zirconia (YSZ). The composition of the second dental component is not limited to high-strength ceramics, although the invention is described herein in relation to high strength ceramic components. Other types of materials that may comprise a dental component to which a porcelain-based dental component can be attached are also intended to be encompassed herein.

The present invention provides for a dental restorative comprising a modified porcelain and a ceramic, second dental component. In a typical ceramic/porcelain dental restoration, there are two ceramic (e.g., YSZ) interfaces: a resin-bonded interface (attaching the ceramic implant to the underlying tooth structure, typically by means of a resin cement), and the veneer interface (attaching the ceramic implant to a porcelain veneer attached thereto). Although superior in terms of mechanical performance (e.g., strength, toughness, and/or fatigue resistance), a consistent problem associated with high-strength ceramics such as zirconia (e.g., YSZ) as the ceramic implant component is poor adhesion due to chemical bonding with a variety of substrates (synthetic or tissue) that can be encountered in dental or other biomedical applications. While there has been much discussion in the scientific literature and commercial development centered on the resin-bonded interface with zirconia, the majority of clinical failures are attributed to chipping or fractures associated with the zirconia/porcelain interface.

Such ceramic-based restorations are generally prepared by attaching the second dental component (e.g., to an existing, underlying tooth structure), which can then be coated with various additional layers. In some embodiments, a dental implant (or another type of dental component) is coated with one or more porcelain veneering layers/glazed bonding layers, e.g., to ensure a natural look to the ceramic-based restoration. For example, a dental component can be coated with a modified porcelain material and fired to give a porcelain overlying a second dental component. Any number of subsequent applications of porcelain material (unmodified and/or modified as described herein) and firings can be performed, e.g., to achieve the desired color and opacity. The methods by which the porcelain-based dental component can be modified may, in certain embodiments, be readily implemented by slight modifications to existing protocols for all-ceramic restoration placement.

The inventors have observed that, following the application and firing of one or more unmodified layers of porcelain veneer onto certain stabilized ceramic components, the porcelain veneer may comprise one or more microcrystalline defects/inclusions. It is believed that these microcrystalline defects result from the migration of certain components of the stabilized ceramic component into the porcelain veneer during high temperature processing (i.e., firing). For example, in specific embodiments, it is believed that yttrium leaches from the surface of a YSZ ceramic dental component and the leached Y ions can react directly with a veneering porcelain to form such microcrystalline inclusions.

As an example, bars (2×3×20 mm in size) were cut from fully sintered YSZ plates (LAVA, 3M ESPE) and ultrasonically cleaned in deionized water. Veneering porcelain (VITAVM 9 Effect Bonder, Vident) was applied to one surface and the coated bars were fired under the manufacturer's recommended protocol (80° C./min ramp to 980° C., soak for 1 min, and cool in vacuum for 6 min). FIG. 2 shows a scanning electron microscopy (SEM) image of the resulting, polished YSZ-porcelain interface, showing micron-sized inclusions (pointed out by the arrows). These inclusions were confirmed by energy dispersive spectrometry (EDAX) analysis as comprising a mixture of Y, Si, and O. Transmission electron microscopy (TEM) studies further indicated that these inclusions are crystalline features within an amorphous porcelain and selected electron diffraction identified them specifically as yttrium-silicate (Y₂SiO₅). The temperature at which these inclusions can form can vary. Thermodynamically, yttrium metal mixed with pure silica will form crystalline yttrium-silicate at a temperature as low as about 800° C. The combinations of yttrium ion diffusion in the YSZ, surface accumulation referenced above, and the high temperature processing of the veneering porcelain, enable these silicate inclusions to form near the interface and throughout the veneering layer.

FIGS. 3A and B illustrate an exemplary fracture surface of a ZirPress/ZirCAD crown originating near a cusp on the occlusal surface. FIG. 3A is an SEM image of the remaining part of the crown and FIG. 3B is a micro-CT scan of the chipped off cusp. The fracture origin is indicated by the arrows in FIGS. 3A and 3B and these figures demonstrate the presence of inclusions throughout the fracture surface. Further SEM analysis of these samples revealed the presence of micron-sized inclusions in the porcelain, which are understood to weaken the glassy matrix and preliminary TEM analysis has indicated a change in the YSZ crystal structure near the ceramic/porcelain interface, consistent with the formation of yttrium silicates, as described above. Although not intending to be limited by theory, it is believed that such yttrium migration may destabilize the tetragonal zirconia at the interface, leading to phase transformation and inducing residual tensile stress in the porcelain layer.

According to the present disclosure, although inclusions and defects are typically considered to be a negative feature of dental materials, it has surprisingly been found that the inclusions described herein can beneficially be incorporated in dental materials. Although small concentrations of such inclusions may be detrimental to the strength and integrity of a porcelain-coated ceramic, the materials described herein, which contain a relatively high content of such inclusions, unexpectedly exhibit greater fracture resistance.

Such ceramic-based restorations can be provided, for example, by using a modified porcelain component incorporating one or more types of metal additives. For example, any metal additive, wherein the metal is capable of forming a crystalline silicate phase, can be used for this purpose. In certain embodiments, the additive can be a metal, a metal compound, or a metal salt. One exemplary metal additive, as demonstrated herein, is yttrium, which may be incorporated into the porcelain in the form of, e.g., YF₃.

The incorporation of small amounts of various additives within porcelain materials is known. For example, small amounts of certain additives are commonly provided to alter the coefficient of thermal expansion of the porcelain material, rendering it more capable of bonding to a ceramic component to which the porcelain is applied. Briefly, porcelains are applied in layers and are fired at high temperatures after each layer. It is generally believed that thermal contraction mismatch at the porcelain-ceramic interface can lead to residual thermal stress upon rapid cooling and that this stress extends all the way from the interface to the occlusal surface.

One way of addressing this concern is to apply a binder/sleeve component directly to the ceramic component prior to the addition of the one or more porcelain layers. The binder/sleeve component may have a slightly different formulation than the subsequent porcelain layers applied thereto, as it is designed to enhance the bonding between the ceramic component and the one or more additional porcelain layers. The enhanced bonding can be provided by incorporating a small amount of one or more additives capable of modifying the coefficient of thermal expansion. These binder/sleeve components, lying between a ceramic component and one or more porcelain layers, are not recognized as providing any function other than modifying the coefficient of thermal expansion of the porcelain to promote bonding between the ceramic component and the one or more porcelain layers.

The present disclosure recognizes an unexpected advantage to modifying a porcelain material to introduce one or more additives capable of forming a crystalline silicate phase within the fired porcelain material. The additive can be anything capable of achieving such an effect (and is thus generally referred to herein as a “crystalline silicate-forming additive”). Generally, the crystalline silicate-forming additive is a metal-containing additive, including but not limited to, an yttria-containing additive, an aluminum-containing additive, a sodium-containing additive, a calcium-containing additive, a magnesium-containing additive, a potassium-containing additive, an iron-containing additive, or a combination thereof. The crystalline silicate-forming additives can be, in certain examples, salts, wherein the metal can readily be released as a free metal ion and incorporated within a silicate structure. Exemplary salts include, but are not limited to, halide salts (e.g., fluoride salts and chloride salts), sulfate salts, carbonate salts, and the like. One exemplary crystalline silicate-forming additive is YF₃. In some embodiments, the crystalline silicate forming additives can be elemental metal powders (e.g., including, but not limited to, yttrium (Y), zirconium (Zr), titanium (Ti), magnesium (Mg), calcium (Ca), aluminum (Al), cerium (Ce), and combinations thereof).

Generally, porcelain materials are prepared by first blending the porcelain components (e.g., ceramic precursors such as silica, alumina, feldspar, calcium carbonate, sodium carbonate, potassium carbonate, and other components as described above). Advantageously, the components are blended in finely divided powder form. The resulting mixture is heated and fused at an elevated temperature (e.g., at least about 1200° C.) to form a glass (also known as a “frit”). The molten glass is quenched, dried, and ground to provide the porcelain material in the form of a powder. The porcelain powder may further comprise various additional components including, but not limited to, binders, pigments, and/or opacifiers, which can be added alongwith the ceramic precursors or can be combined with the porcelain powder.

Variations in the chemical makeup of the porcelain powder can impact the physical properties of the ceramic precursor mixture and thus may dictate the methods that are used to use the ceramic powder. For example, certain ceramic powders may require different temperatures to fuse the particles following application to a substrate. Porcelains can be characterized as “high-fusing ceramics” (generally having a fusion temperature of from about 1288° C. to about 1371° C.), “medium-fusing ceramics” (generally having a fusion temperature of from about 1093° C. to about 1260° C., or “low fusing ceramics” (generally having a fusion temperature of from about 660° C. to about 1066° C.).

Various porcelain powders are commercially available, including, but not limited to, IPS Empress® layering materials and IPS e.max® Ceram (Ivoclar Vivadent, Amherst, N.Y.); Ceramco® porcelains (Dentsply Prosthetics, York, Pa.); Noritake Super Porcelain EX-3, TI-22, Cerabien, or Cerabien ZR (Noritake Dental Supply Co., Ltd., Japan); OPC® Low Wear™ (Jeneric/Pentron Inc., Wallingford, Conn.); Vita Titanium Porcelain, VMK, VM®7, VM®9, and VM®13 porcelains (Vident, Brea, Calif.); Pulse, Creation, and Authentic Powders (Jensen Dental, North Haven, Conn.); CeraMax (AlphaDent Co., Ltd., Korea); C-Mix Fine Grain Porcelain (Arro Rosenson, Inc., Mineola, N.Y.); Duceram, Duceragold™, Cercon® Ceram Kiss, and Allceram veneering ceramics (DeguDent GmbH, Germany); Lava™ Ceram Overlay Porcelain (3M ESPE); pulse® ceramics (Zubler USA); ISIS™ porcelain (Provident Dental Products, Somerset, N.J.); and Synspar® and Avante® porcelains (Pentron® Ceramics, Inc., Somerset, N.J.). Other exemplary porcelains and methods for their production are described, for example, in U.S. Pat. Nos. 4,645,454 to Amdur et al.; 4,741,699 to Kosmos et al.; 5,281,563 to Komma et al.; 5,453,290 to Van der Zel; 5,944,884 to Panzera et al.; and 6,428,614 to Brodkin et al.; and U.S. Patent Application Publication Nos. 2007/0196788 and 2009/0298016 to Chu et al., which are all incorporated herein by reference.

According to the invention, one or more such porcelain powders are mixed with one or more crystalline silicate-forming additives, as described herein, to provide a modified porcelain powder. The one or more crystalline silicate-forming additives can be included as a porcelain component along with the other components of the porcelain, such that all components are heated, fused together, cooled, and ground to give a modified porcelain powder or the one or more additives can be added as additional components following formation of the porcelain powder. “Modified porcelain powder” as used interchangeably herein, can encompass various modified materials containing varying levels of crystalline silicate-forming additives (i.e., one or more additives capable of forming a crystalline silicate phase within a fired porcelain material). The amount of crystalline silicate-forming additives added to the porcelain can vary widely, e.g., between about 1% and about 80% by weight based on the porcelain powder (comprising the one or more additives and the remaining porcelain components). In some embodiments, the amount of crystalline silicate-forming additives added to the porcelain powder can be between about 5% by weight and about 50% by weight of the porcelain powder, and preferably between about 10% and about 40% by weight of the porcelain powder. In certain embodiments, the amount of metal (based on the addition of metal-containing crystalline silicate-forming additives) incorporated within the porcelain powder can be between about 5% by weight and about 50% by weight of the porcelain powder, e.g., about 5% by weight or more, about 10% by weight or more, about 15% by weight or more, about 20% by weight or more or about 30% by weight or more of the porcelain powder.

Generally, porcelain-based dental materials are prepared by providing one or more porcelain powders and mixing the one or more porcelain powders prior to application with a solvent (e.g., distilled water or an inorganic or organic liquid, such as an alkyl polyhydric alcohol, aryl alcohol, diaryl ether, or a derivative or combination thereof; and/or methacrylate monomers) to give a slurry. The consistency of the slurry can vary, and may be, for example, in the form of a paste. Various other additives can be included within the slurry, for example, to adjust the consistency or drying process (e.g., working time) of the slurry. For example, glycerine, propylene glycol, and/or alcohols are common additives. According to the invention, the one or more crystalline silicate-forming additives can be incorporated within the slurry (rather than being incorporated within the porcelain powder prior to slurry formation) to give a modified porcelain slurry.

Regardless of the stage at which the one or more crystalline silicate-forming additives are incorporated within the porcelain, in certain embodiments, the content of crystalline silicate-forming additives with respect to the amount of porcelain is preferably about the same. For example, where the crystalline silicate-forming additives are added directly to the slurry (rather than included as a component of the porcelain powder), the amount of crystalline silicate-forming additive in the slurry may still be, e.g., between about 5% by dry weight and about 50% by dry weight of the porcelain slurry, and preferably between about 10% and about 40% by dry weight of the porcelain slurry.

The resulting additive-containing slurry can then be applied to the surface of a second dental component (e.g., a core) in various ways. For example, it may be applied by brushing, spatulation, spraying, dipping, whipping, vibrating, and/or electrodeposition onto the second dental component. The coated second dental component is then fired to sinter the porcelain coating, which generally removes the solvent as well. The temperature required for sintering can vary; as noted above, the fusion temperatures of different porcelain compositions can vary widely. Generally, with commercially available porcelain powders, the manufacturer provides guidance on the necessary time and temperature for sufficient sintering. According to the present invention, it may be necessary to adjust these parameters to account for the presence of the one or more additives, and these adjustments would be well within the abilities of one of ordinary skill in the art.

In some embodiments, use of certain types of solvents and/or additives in the slurry can facilitate the preparation of the porcelain layers. For example, the solvent can comprise a polymerizable resin (i.e., comprising monomers) that is self-curing or light-cured. Following application of the porcelain slurry to the second dental component, the polymerizable resin can be cured to fix the porcelain coating in place in the desired shape and thickness. The dental structure can then be fired, which results in removal of the cured resin and sintering of the porcelain coating.

The thickness of each layer can vary. Further, multiple layers of porcelain slurry are generally applied to the second dental component to give a multilayered dental structure. Varying layers can be added to the dental component, e.g., at least about 2, such as between about 2 and about 5. In some embodiments, a greater number of layers may be required, e.g., to achieve the desired aesthetic appearance. The one or more layers, after application, are generally fired (e.g., after each layer is applied).

The multiple porcelain layers may be the same or different. For example, for some applications, layers of varying translucencies and/or colors can be applied so as to produce a prosthetic or veneer that closely resembles actual tooth. In some embodiments, the general composition of the porcelain is comparable in the multiple layers, with slight variations in the amount of pigment and/or opacifying material. According to the invention, some layers may comprise the one or more crystalline silicate-forming additives, whereas others may not comprise the one or more crystalline silicate-forming additives. Advantageously, according to the invention, two or more of the porcelain layers applied to the second dental component comprise one or more crystalline silicate-forming additives, as a greater concentration of crystalline silicate-forming additives in the porcelain component as a whole will lead to a beneficial greater concentration of crystalline inclusions following firing of the restoration.

Preferably, a majority of the layers, e.g., nearly all of the layers, comprise the one or more crystalline silicate-forming additives. Advantageously, incorporating the one or more crystalline silicate-forming additives in a majority of the layers can lead to a porcelain component having a relatively uniform distribution of the one or more crystalline silicate-forming additives. Such a component advantageously comprises the one or more crystalline silicate-forming additives dispersed throughout the thickness of the porcelain component (e.g., substantially from the porcelain/ceramic interface to the occlusal surface). It is noted that larger inclusions and/or a higher concentration of inclusions will occur in some embodiments, e.g., for multiple crystalline silicate-forming additive-containing porcelain veneering layers and/or for porcelain slurries containing a high concentration of crystalline silicate-forming additives.

Other porcelain-based dental materials are prepared by compacting one or more porcelain powders in solid form (“pressable” porcelains, or “press-to” ceramics). Exemplary commercially available pressable porcelain powders include, but are not limited to, Cergo® Kiss, Cercon® Ceram Press, Ducera® Press (DeguDent GmbH, Germany); IPS e.max Press and Empress® ceramics (Ivoclar Vivadent, Amherst, N.Y.); and Finesse® (Dentsply Prosthetics, York, Pa.). According to the invention, one or more pressable porcelain powders are mixed with one or more crystalline silicate-forming additive to provide a modified pressable porcelain powder. The modified ceramic powder is subjected to pressure and heat, which converts the powder to a viscous state. The powder is pressed into the desired form and cooled. The powder can be cooled on a frame, such as on the second dental component of the present invention, to give a composite dental structure. For details on processing conditions and press ceramics, see, for example, EP 0231 773 and U.S. Patent Application Publication No. 2009/0011916 to Steidl, which are incorporated herein by reference.

Following application of one or more crystalline silicate-forming additive-containing porcelain layers to a ceramic core and firing of the one or more porcelain layers thereon, the porcelain component as a whole is noted to comprise crystalline silicate inclusions. The crystalline inclusion content in a final dental restorative based on the methods provided herein can vary, depending, for example, on the concentration of crystalline silicate-forming additive in the porcelain powder, the number of layers of porcelain coating applied to the ceramic core, and the makeup of the ceramic core. It is noted that, where a stabilized ceramic core is used, the overall restorative may comprise inclusions arising from both the additive-doped porcelain powder slurry and inclusions arising from the migration of certain ions (e.g., Y ions) from the stabilized ceramic core into the porcelain component. Inclusions arising from migration of ions from the stabilized ceramic core can, in some embodiments, be present throughout the thickness of the porcelain, as shown in FIG. 4. FIG. 4A is a micro-CT scan (cross-section) of a veneered YSZ plate (veneered with a ceramic that does not comprise any added yttrium). The image shows white spots (with arrows pointing to certain exemplary spots) that were confirmed to be crystalline structures (specifically, yttrium silicate inclusions). The dark spots indicate pores in the porcelain. It is clear based on this image that the yttria has migrated through the thickness of the porcelain veneer and has formed silicate precipitates through the full thickness. FIG. 4B is an SEM cross-section of the same sample, displaying areas of migrated Y that have formed crystalline defects, as confirmed by EDS (light areas indicated by arrows).

In some embodiments, as noted briefly above, the ceramic second dental component can be a pre-treated component that has been pre-treated to give, e.g., a substrate surface comprising a fluorinated metal oxide. For details on the methods of treatment to provide such a surface, see PCT Int. App. Pub. No. WO/2012/054702 and PCT Int. App. Pub. No. WO/2013/158836, both to Piascik et al., which are incorporated herein by reference. In such embodiments, it has been noted that the fluoride treatment can limit the ability of Y to migrate from a YSZ core out to the porcelain layer through the YSZ/porcelain interface. In such embodiments, consequently, Y-containing inclusions are not expected to form within an unmodified porcelain material (as little to no Y diffuses into the porcelain).

Therefore, modifying the porcelain component to add crystalline silicate-forming additives as described herein may be unnecessary to combat the negative effects of failure-inducing inclusions as they are likely to not be formed in such restorations. However, in some embodiments, it may be desirable to modify the porcelain component to add crystalline silicate-forming additives as described herein even where inclusions due to diffusion are not expected to be formed. Specifically, the specific formation of inclusions according to the methods herein can still strengthen the porcelain component, in some embodiments, not only with respect to a porcelain component having a low concentration of inclusions, but also with respect to a porcelain component having little to no inclusions.

In various embodiments as described herein, the porcelain veneering can comprise a plurality/high density of metal silicate crystalline phase inclusions, preferably distributed throughout the thickness of the porcelain material. Advantageously, the total metal additive content within the porcelain layer of a fully fired dental restorative (including both metal additives from the modified porcelain slurry and metal that has leached from a stabilized ceramic core to which the slurry is applied) can be, for example, between about 2% and about 20% by weight, e.g., between about 5% and about 15% by weight.

Advantageously, according to the present disclosure, a sufficient number of inclusions are generally provided to strengthen the porcelain material as compared with an unmodified porcelain. As illustrated in FIG. 5, a crack initiation site is present in schematic A, where a single inclusion is contained within the top (veneering ceramic) layer. In contrast, in schematic B, a plurality of inclusions are provided, which should inhibit the initiation and/or propagation of cracks within the ceramic layer and strengthen the ceramic veneering component and/or the ceramic restorative as a whole.

Advantageously, in some embodiments, such inclusions are present throughout the thickness of the porcelain component (i.e., from the surface adjacent to the ceramic core component through the thickness of the porcelain component to the exposed surface). The inclusions can vary in size and shape. In certain embodiments, the largest dimension of such inclusions is on the order of from about 0.5 microns to about 10 microns. The number of inclusions that is sufficient to strengthen the porcelain veneering layer can vary. In some embodiments, the porcelain may comprise at least about 2% crystalline inclusions by volume, at least about 5% crystalline inclusions by volume, at least about 10% crystalline inclusions by volume, at least about 15% crystalline inclusions by volume, or at least about 20% crystalline inclusions by volume. For example, the porcelain may comprise between about 2% and about 60% crystalline inclusions by volume, such as between about 5% and about 40% crystalline inclusions by volume.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of increasing the integrity of a dental restorative, comprising:
providing a dental component;
applying a porcelain slurry comprising about 5% or more by weight of an additive capable of forming a crystalline silicate phase to at least a portion of the surface of the dental component to give a coated dental component; and
firing the coated dental component.

2. The method of claim 1, wherein the dental restorative comprises a crown, bridge, veneer, inlay, or onlay.

3. The method of claim 1, wherein the dental component comprises an abutment.

4. The method of claim 1, wherein the porcelain slurry comprises about 10% or more by weight of an additive capable of forming a crystalline silicate phase.

5. The method of claim 1, wherein the additive comprises a metal, a metal compound, or a metal salt.

6. The method of claim 1, wherein the additive comprises YF₃.

7. The method of claim 1, wherein the dental component comprises a tetragonally stabilized ceramic.

8. The method of claim 1, wherein the dental component comprises a yttria-stabilized ceramic.

9. The method of claim 1, wherein the dental component comprises zirconia, alumina, titania, or chromium-oxide.

10. The method of claim 1, wherein each of the applying and firing steps is performed at least two times.

11. The method of claim 1, further comprising treating the dental component with a fluorine-containing reagent prior to the applying step.

12. A dental restorative comprising a dental component and a modified porcelain coating overlying at least a portion of the dental component, wherein the modified porcelain coating comprises a plurality of crystalline inclusions present in an amount of at least about 5% by volume of the porcelain coating.

13. The dental restorative of claim 12, wherein the dental restorative comprises a crown, bridge, veneer, inlay, or onlay.

14. The dental restorative of claim 12, wherein the dental component comprises an optionally stabilized ceramic selected from the group consisting of zirconia, alumina, titania, and chromium-oxide.

15. The dental restorative of claim 12, wherein the dental component comprises an abutment.

16. The medical restorative of claim 12, wherein the crystalline inclusions comprise metal silicate inclusions.

17. The medical restorative of claim 12, wherein the crystalline inclusions comprise yttrium silicate inclusions.

18. A dental restorative comprising a yttria-stabilized zirconia dental component and a modified porcelain coating overlying at least a portion of the dental component, wherein the modified porcelain coating comprises a plurality of yttrium silicate inclusions present in an amount of at least about 2% by volume of the porcelain coating.

19. The dental restorative of claim 18, wherein the dental restorative comprises a crown, bridge, veneer, inlay, or onlay.

20. The dental restorative of claim 18, wherein the dental component comprises an abutment.
